(19) United States
(12) Patent Application Publication
(10) Pub. No.: US 2024/0117872 A1
(43) Pub. Date: Apr. 11, 2024

(54) METHOD FOR MONITORING A GEARBOX IN AN AIRCRAFT, MONITORING SYSTEM FOR A GEARBOX IN AN AIRCRAFT, AND AIRCRAFT HAVING THE MONITORING SYSTEM

(71) Applicant: AIRBUS HELICOPTERS TECHNIK GMBH, Calden (DE)

(72) Inventors: Sergej LIEDER, Kassel (DE); Timm ROESENER, Kaufungen (DE); Joerg LITZBA, Fuldatal (DE)

(73) Assignee: AIRBUS HELICOPTERS TECHNIK GMBH, Calden (DE)

(21) Appl. No.: 18/276,962

(22) PCT Filed: Feb. 10, 2022
(86) PCT No.: PCT/EP2022/053184

§ 371 Date: Aug. 11, 2023

(30) Foreign Application Priority Data
Feb. 11, 2021 (DE) 102021201290.6

(51) Int. Cl. F16H 57/01; G01N 33/28

(52) U.S. Cl. CPC F16H 57/01; G01N 33/2858; B64D 2045/0085

(57) ABSTRACT

A method for monitoring a gearbox in an aircraft. The gearbox comprises at least one gearbox section with at least one measuring area. A first and a second electrical contact are arranged in the measuring area at a spacing from one another. A magnetic field is generated in the gearbox section. The magnetic field is adapted to move at least one ferromagnetic particle arranged in the gearbox section towards the measuring area, so that a bridge produced from one or more of the ferromagnetic particles is formed between the two electrical contacts. A measured value is generated based on an electrical parameter between the two electrical contacts. A measure of the criticality of a flight operation is determined on the basis of the measured value.

The invention relates to a method for monitoring a gearbox in an aircraft with the features of the preamble of claim 1. The invention further relates to a monitoring system for a gearbox in an aircraft as well as to an aircraft with the monitoring system.

High torques can occur in gearboxes in aircraft or helicopters and can cause wear or natural abrasion on the gearbox components. It is therefore necessary to monitor the condition of the gearbox in order to ensure flawless and safe flight operations. To this end, sensors for detecting ferromagnetic particles are known wherein a magnetic field is generated in order to attract the ferromagnetic particles and to detect them by means of a suitable detector.

As an example, the document DE 1020 12219242 A1 discloses a measuring apparatus for detecting ferromagnetic particles which are movably arranged in a liquid volume, wherein the measuring apparatus comprises a magnetic means for generating a magnetic field in the liquid volume, a measuring means for the total electrical resistance of the liquid volume with the ferromagnetic particles therein, as well as a means for checking the function of the measuring apparatus with at least one low-value resistor of between 50 and 500 ohms for carrying out a resistance measurement.

The basis of the invention is to propose a method for monitoring a gearbox which provides a qualitative statement about the state of the gearbox. Furthermore, an objective of the invention is to propose a corresponding monitoring system as well as an aircraft with the monitoring system.

This objective is achieved by a method with the features of claim 1, a monitoring system with the features of claim 9 as well as an aircraft with the features of claim 14. Preferred or advantageous embodiments of the invention are defined in the dependent claims, the description below and the accompanying figures.

In the context of the invention, a method for monitoring a gearbox in an aircraft is proposed. The gearbox comprises a gearbox section with at least or exactly one measuring area. Here, the gearbox section can be defined by an interior space of the gearbox or a subspace of the gearbox interior. The measuring area is preferably arranged in an area of an oil sump and/or a bottom area of the gearbox. Alternatively, however, the at least one measuring area can also be arranged at any other point of the gearbox section wherein increased wear or abrasion of the gearbox components is to be expected. Finally, in all cases, the measuring area is arranged in an area wherein a flow of oil takes place. As an example, a volume of oil can be injected into a bearing through an oil nozzle, so that the oil entrains particles as it flows through the bearing, which particles are then supplied to a particle detection sensor by the further flow of oil.

A first and a second electrical contact are arranged in the measuring area, wherein the two electrical contacts are arranged spaced apart from one another. Preferably, the spacing forms a gap between the two contacts, by means of which the contacts are electrically separated from one another. In particular, the two electrical contacts are connected to a current source, wherein the circuit is interrupted in the measuring area. The two electrical contacts can be arranged at a distance of less than 1 mm, preferably less than 0.5 mm, in particular less than 0.1 mm from one another. In addition, alternatively or optionally, individual particles with a size of more than 0.1 mm, preferably more than 0.5 mm, in particular more than 1 mm, can be detected by the electrical contacts. Ultimately, however, the pure particle size is not decisive for successful detection but rather, it is the volume formed by the particles which specifies an electrical resistance and above all enables current to be conducted between the contacts.

The method is characterised by the following steps:
generating a magnetic field in the gearbox section, wherein the magnetic field is configured to move at least one ferromagnetic, in particular electrically conductive, particle arranged in the gearbox section towards the measuring area, so that a bridge, in particular electrically conductive, forms or is formed from one or more of the ferromagnetic particles between the two electrical contacts;
generating a measured value based on an electrical parameter between the two electrical contacts; and
determining a measure of the criticality of a flight operation based on the measured value.

The magnetic field can be generated permanently or temporarily, for example, in particular for the duration of the measurement. Preferably, the magnetic field is generated within the gap. In particular, an operative area of the magnetic field, i.e. the area wherein the ferromagnetic particles are attracted by the magnetic field, is arranged within the measuring area or overlapping the measuring area.

In particular, the bridge is only then formed when one or more of the ferromagnetic particles is arranged in the measuring area. Preferably, during the formation of the electrically conductive bridge, the distance or gap between the two electrical contacts is bridged, so that the circuit is closed and the measured value is generated. In particular, depending on the particle size, the bridge can be formed by precisely one ferromagnetic particle or by an agglomeration of a plurality of the ferromagnetic particles in the measuring area or within the gap. The electrical parameter can in particular be an impedance. In particular, in the case of an arrangement of the at least one ferromagnetic particle between the electrical contacts, a change in the impedance takes place, whereupon it will be possible to infer an amount and/or size of the ferromagnetic particles on the basis of the change in impedance. Preferably, the real part and/or the imaginary part of the impedance is evaluated. It should be noted that the electrical parameter of the capacitance, frequency, conductivity or the like can be detected directly or indirectly.

In particular, the measured value can be evaluated on the basis of an evaluation criterion in order to determine the measure of the criticality. As an example, the evaluation criterion can be defined by a stored nominal value or range of values with which the detected measured value is compared. As an example, in the context of the evaluation, the measured value can be converted into a comparable value. Alternatively, however, the measured value can also be evaluated directly. In particular, on the basis of the measure of the criticality, it can be decided whether there is damage to the gearbox and thus a risk to flight operations.

The advantage of the invention lies in particular in the fact that, on the basis of the measure of the criticality, the pilot is given the possibility of independently being able to make a decision regarding a mission interruption during a mission. In addition, based on the measure of the criticality, a qualitative statement can be made about the state of the gearbox. In addition, the method makes it possible to realize a detection of ferromagnetic particles in the gearbox in a simple and cost-effective manner, wherein at the same time, the number and size of the detected particles can be inferred by an evaluation of the electrical parameter.

In a specific implementation, provision is made for a warning signal to be generated based on the measure of the criticality, wherein a measure of the criticality of the flight operation is indicated based on the warning signal. Preferably, the warning signal is output in real time when a current measured value meets the evaluation criterion. As an example, the warning signal may be output as an electrical signal which is provided for further processing of a computing and/or indicator device. In particular, the measure of the criticality can be indicated optically and/or acoustically and/or haptically. Thus, a danger to flight operations can be communicated to the pilot in a simple manner, whereupon the pilot can make a decision regarding the mission on the basis of the indication.

In a further preferred embodiment, the measure of the criticality is classified into at least or exactly two criticality levels, wherein a respective warning signal is generated for each one of the criticality levels. Information regarding the criticality can preferably be transmitted via the warning signal. In particular, at least one associated evaluation criterion can be defined for each criticality level, which evaluation criterion comprises to be met for a corresponding indication of the criticality to be made. Preferably, the criticality levels are classified on the basis of the particle size and/or number of particles which are determined on the basis of the electrical characteristic value or a change in the electrical characteristic value. By indicating the criticality incrementally, the pilot can easily be shown whether the mission comprises to be broken off or whether the mission can be terminated, or possibly restricted.

In a further specific embodiment, no criticality is indicated in a first criticality level, a low criticality is indicated in a second criticality level, and a high criticality level is indicated in a third criticality level. As an example, the first level is indicated when no particles or only a small number of particles with a small particle size, for example less than 10 micrometres, are detected. As an example, the second level is indicated when one or a few particles with an average particle size, for example greater than 10 micrometres and/or less than 50 micrometres, are detected. As an example, the third level is indicated when at least one particle with a large particle size, for example greater than 50 micrometres, and/or a high number of particles are detected. Due to the different criticality levels, damage to the gearbox can be reliably classified, so that the pilot can assess a risk to flight operations much better.

In a further implementation, the measure of the criticality of the flight operation is rated on the basis of at least one further measured value which is detected independently of the measured value. In particular, the at least one further measured value is detected at the same time as or simultaneously with the measured value and correlated with the measured value, whereupon a rating of the criticality is carried out as a function of the correlated result of the measurement. The rating of the measured value is preferably carried out with the aid of at least one rating criterion. By taking at least one further measured value into account, the reliability of the measurement can be increased and at the same time, a misinterpretation of an individual measured value can be reduced.

In a specific embodiment, a low criticality is indicated when the measured value and/or the further measured value are classified into the second criticality level. In particular, a separate evaluation criterion is stored for each measured value, with the aid of which the measured value is classified into the respective criticality level. Preferably, the measured values are evaluated and/or rated on the basis of decision-making logic. Optionally, the measured values may be weighted differently. As an example, the weighting may be selected as a function of a current operating state and/or of the measuring point and/or of the detected particles. Thus, for example, in the case of detecting particles at typical trouble spots, a high criticality can be indicated even when the further measured value does not meet the associated evaluation criterion or is classified into the first or second criticality level. Furthermore, a high criticality is indicated when the measured value and/or the further measured value is/are classified into the third criticality level. In particular, a high criticality is indicated when at least two or all of the measured values are classified into the third criticality level. Optionally, different weights can also be taken into account. A method is therefore proposed which compares and rates different measured values with one another, so that the measurement reliability is significantly increased.

In a further specific embodiment, the further measured value is output when at least one further ferromagnetic particle is detected in a further measuring area in the gearbox. In particular, the further measuring area can be arranged in the gearbox section or in a further gearbox section. Preferably, ferromagnetic particles are detected in a further measuring area, in a manner that comprises already been described above. In particular, detection of ferromagnetic particles can take place in at least or exactly two, preferably more than three, in particular more than five different positions or measuring areas in the gearbox. In addition, alternatively or optionally, the further measured value is output in the event of a change in a gearbox parameter. As an example, one or more known gearbox parameters such as vibrations, noise, rotational speed, torque, gearbox temperature or the like may be evaluated for this purpose. Thus, a method is proposed which is characterised by a high measurement accuracy, wherein a failure of the gearbox can be detected early and reliably by taking a plurality of measured values into account.

Preferably, the electrical parameter is an ohmic resistance. In particular, particles with a small cross section have a higher resistance than particles with a larger cross section. As an example, the ohmic resistance can be determined using Ohm's law and provided as a measured value. The particle size can therefore be determined on the basis of the ohmic resistance. In addition, alternatively or optionally, the electrical parameter is an electrical voltage. In particular, a voltage drops across the electrically conductive bridge, so that the voltage difference can be used to form the measured value. As an example, the electrical voltage can be determined based on Ohm's law. The particle size can therefore be determined on the basis of the voltage or a change in the voltage.

The invention further concerns a monitoring system which is configured and/or suitable for monitoring a gearbox in an aircraft. In particular, the monitoring system is configured and/or suitable for carrying out the method as comprises already been described above.

The monitoring system comprises at least or exactly one particle detection sensor which is configured to detect ferromagnetic particles in a measuring area of the gearbox. In particular, the particle detection sensor is arranged in the gearbox section of the gearbox, wherein the particle detection sensor defines the measuring area. In this regard, the measuring area should be understood to be an area around the sensor within which the ferromagnetic particles are detected. To this end, the particle detection sensor comprises two electrical contacts which are spaced apart from one another and which are arranged within the measuring area and are connected to an electrical circuit. The particle detection sensor is preferably configured as a chip detection sensor which is configured and/or suitable for detecting ferromagnetic and in particular metallic chips. Such sensors are also known, inter alia, as magnetic abrasion detectors (magnetic chip detectors).

The particle detection sensor comprises a magnet which is configured and/or suitable for generating a magnetic field. In this regard, the magnet can be configured as a permanent magnet or as an electromagnet. The magnet comprises the function of moving one or more ferromagnetic particles arranged in the gearbox interior into the measuring area of the particle detection sensor, so that an electrically conductive bridge is formed between the two electrical contacts from one or more of the ferromagnetic particles. The magnet is preferably arranged in such a manner that an operative area of the magnet wherein the ferromagnetic particles are magnetically attracted is arranged between the two contacts and/or in the measuring area. To this end, for example, the magnet can be arranged in the gap between the two contacts. In the presence of one or more of the ferromagnetic particles between the electrical contacts, it is possible to detect between the two contacts on the basis of the electrical parameter. In this regard, the particle detection sensor is configured to output a measured value on the basis of a measured electrical parameter. In particular, the measured value can be output as an electrical measurement signal.

The monitoring system also comprises an evaluation device which is configured and/or suitable for evaluating the measured value in order to determine a measure of the criticality of the flight operation by evaluating the measured value. Preferably, the particle detection sensor and the evaluation device are connected to each other for the purposes of signalling. In particular, at least one evaluation criterion is stored in the evaluation device, with the aid of which the measured value is mapped into the measure of the criticality. As an example, the evaluation device can be configured as a hardware or software module in a flight computer. The evaluation device is configured.

In a further embodiment, the monitoring system comprises at least or exactly one further particle detection sensor, which is configured to detect ferromagnetic particles in a further measuring area of the gearbox. In particular, the measuring area and the further measuring area are arranged at different positions of the gearbox, in particular in different and/or separate sections of the gearbox. Alternatively, however, the two measuring areas can also be arranged adjacent to one another, in particular in a common gearbox section. To this end, the further particle detection sensor is optionally arranged in the gearbox section or the further gearbox section. In particular, the particle detection sensor and the further particle detection sensor are of identical construction. As an example, the monitoring system may have more than two, in particular more than four, in particular more than six of the particle detection sensors. The further particle detection sensor is configured to output a further measured value when at least one further ferromagnetic particle is detected. Preferably, all of the particle detection sensors are connected to the evaluation device for the purposes of signalling. The evaluation device is configured to rate a measure of the criticality of the flight operation based on the measured value and the further measured value. In particular, the evaluation device is configured to rate the criticality with the aid of decision-making logic.

In an alternative or optional supplementary embodiment, the monitoring system comprises at least one or exactly one gearbox monitoring sensor which is configured and/or suitable for detecting a gearbox parameter. In principle, the gearbox monitoring sensor can be configured as a temperature sensor, rotational speed sensor or torque sensor. However, the gearbox monitoring sensor is preferably configured as an acceleration sensor which is suitable for detecting vibrations in the gearbox. The gearbox monitoring sensor is configured to output the gearbox parameter as a further measured value and to transmit it to the evaluation unit. To this end, the gearbox monitoring sensor and the evaluation device are connected to each other for the purposes of signalling. The evaluation device is configured to rate a measure of the criticality of the flight operation by evaluating the electrical measured value and the gearbox parameter. In particular, the sensor data from a plurality of gearbox monitoring sensors can be used and correlated with the measured values for the at least one particle detection sensor in order to rate the criticality.

In a further embodiment, the monitoring system comprises an indicator device which is configured to indicate a measure of the criticality of the flight operation. In particular, the indicator device is arranged and/or can be arranged in the pilot's field of vision, preferably inside the cockpit. The indicator device is preferably configured as an illuminant or a display which can take up at least two different indicator states for indicating the criticality. As an example, the indicator states may be distinguished from one another by indicating different light signals and/or messages.

In a specific embodiment, a V-shaped gap is formed between the two contacts, which gap is configured to accumulate a plurality of the ferromagnetic particles. In particular, the two contacts are arranged at an angle of more than 20 degrees, preferably more than 50 degrees, in particular more than 80 degrees with respect to each other. Because of the V-shaped gap, ferromagnetic particles of different sizes can be accumulated. This means that during a measurement over a period of time, it is possible to infer normal abrasion or critical wear.

In a further aspect, the invention concerns an aircraft with the monitoring system as comprises already been described above. The aircraft can in principle be configured as an aircraft, an air taxi or the like. Preferably, however, the aircraft is configured as a helicopter.

Further features, effects and advantages of the invention will become apparent from the following description of a preferred exemplary embodiment of the invention and the accompanying figures, in which:

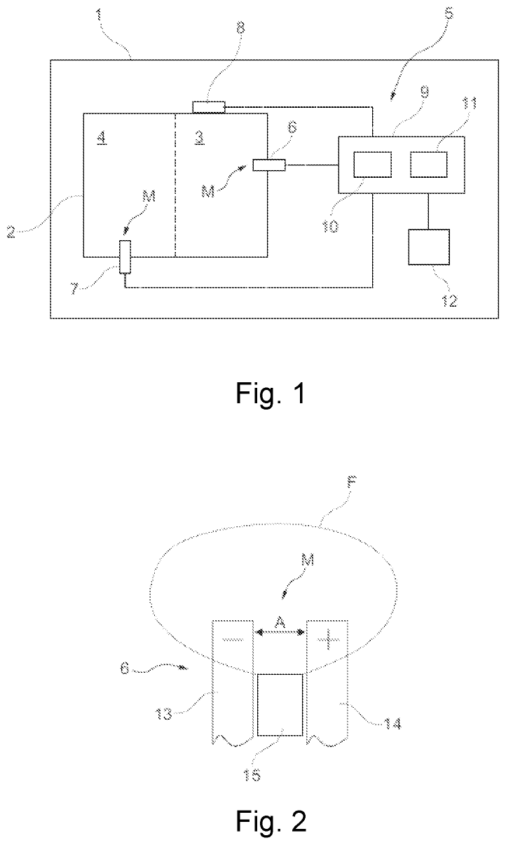

FIG. 1 shows a highly diagrammatic representation of an aircraft with a monitoring system as an exemplary embodiment of the invention;

FIG. 2 shows a diagrammatic illustration of a particle detection sensor of the monitoring system according to FIG. 1;

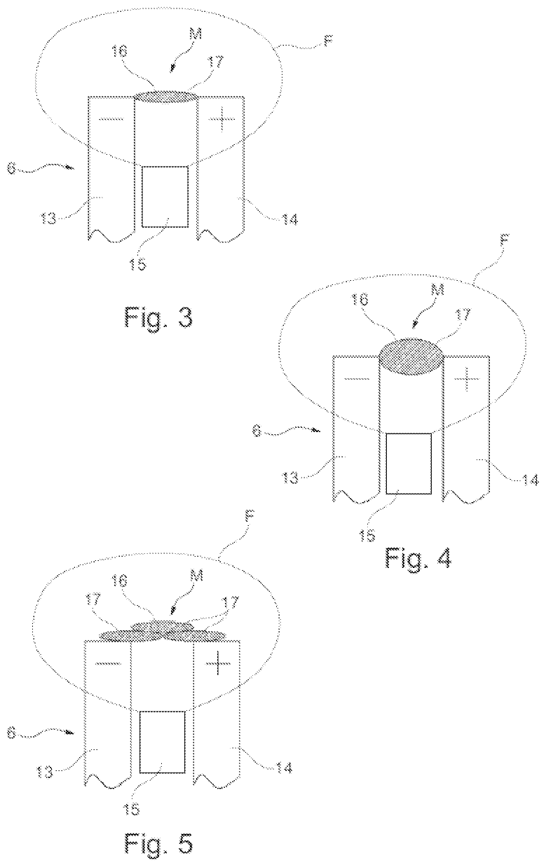

FIG. 3 shows the particle detection sensor in the same representation as in FIG. 2, in a first detection state;

FIG. 4 shows the particle detection sensor in the same representation as in FIG. 2, in a second detection state;

FIG. 5 shows the particle detection sensor in the same representation as in FIG. 2, in a third detection state; and

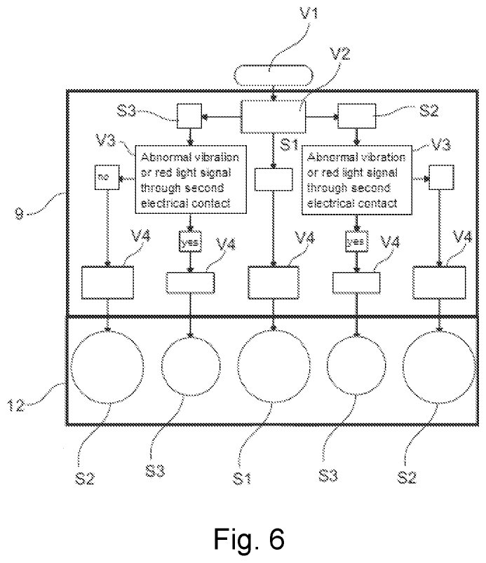

FIG. 6 shows decision-making logic for rating a criticality of the flight operation with the aid of a diagrammatic block diagram.

FIG. 1 shows a highly diagrammatic block diagram of an aircraft 1, shown here only as a functional block, as an exemplary embodiment of the invention. As an example, the aircraft 1 is configured as a helicopter.

The aircraft 1 comprises a gearbox 2 which serves for the gearbox of a drive torque. As an example, the gearbox 2 can transfer the drive torque from an engine to a power unit and/or to one or more rotors.

The gearbox 2 is preferably configured as a mechanical gearbox, for example a toothed wheel gearbox, which is exposed to high torques and rotational speeds during flight operation.

The gearbox 2 can be subdivided into several gearbox sections 3, 4, which are configured, for example, as sub-sections of an interior space of the gearbox 2. As an example, the gearbox sections 3, 4 can be spatially separated from one another, for example as wet and dry chambers. Alternatively, however, the gearbox sections 3, 4 may also define a common interior space of the gearbox 2.

The aircraft 1 comprises a monitoring system 5 which is used to monitor the gearbox 2 during flight operations. To this end, the monitoring system 5 comprises at least one particle detection sensor 6 which is used to detect ferromagnetic particles. These ferromagnetic particles can form as a result of abrasion, for example, or also as a result of damage to the gearbox components due to the high loads. In this regard, the particle detection sensor 6 is arranged in the first gearbox section 3 in order to detect any ferromagnetic particles arranged therein. In this regard, the particle detection sensor 6 outputs a measured value when one or more ferromagnetic particles are detected.

Optionally, the monitoring system 5 may have a further particle detection sensor 7 which is arranged in the second gearbox section 4 for detecting ferromagnetic particles. By means of the further particle detection sensor 7, ferromagnetic particles can therefore be detected at different positions in the gearbox 2. In this regard, the further particle detection sensor 7 outputs a further measured value when one or more ferromagnetic particles are detected.

In addition, alternatively or optionally, the monitoring system 5 may have a gearbox monitoring sensor 8, which is used to detect a gearbox parameter. As an example, the gearbox monitoring sensor 8 is configured as an acceleration sensor which is arranged in or on the gearbox 2 in order to detect vibrations. In this regard, the gearbox monitoring sensor 8 outputs the gearbox parameter as a further measured value.

The monitoring system 5 comprises an evaluation device 9 which is configured to evaluate the measured values. To this end, the sensors 6, 7, 8 are connected to the evaluation device 9 for the purposes of signalling. As an example, the evaluation device 9 forms an integral component of a flight computer.

The evaluation device 9 comprises an evaluation module 10, wherein the evaluation module 10 is configured to evaluate the measured values on the basis of at least one evaluation criterion in order to determine a measure of the criticality of the flight operation. Furthermore, the evaluation device 9 may have a rating module 11 which is configured to correlate the measured values from the sensors 6, 7, 8 with one another and to rate the criticality on the basis of a rating criterion.

The evaluation device 10 is configured to output a warning signal if at least one evaluation criterion and/or rating criterion is fulfilled, wherein the warning signal is provided to an indicator device 12. The indicator device 12 may, for example, be configured as an illuminant, wherein the indicator device 13 converts the warning signal into a light signal in order to indicate the measure of the criticality of the flight operation. To this end, the indicator device 10 is preferably arranged in the pilot's field of vision, whereupon the pilot decides on the basis of the light signal whether the mission comprises to be interrupted or whether the mission can be terminated.

In a diagrammatic representation, FIG. 2 shows the particle detection sensor 6 as a further exemplary embodiment of the invention. It should be noted that the following description of the function can be analogously applied to the further particle detection sensor 7.

The particle detection sensor 6 comprises a first and a second electrical contact 13, 14, which are electrically separated from one another by a spacing A and are arranged within the respective gearbox section 3, 4. The two contacts 13, 14 are connected to an electrical circuit which is interrupted in the area of the contacts 13, 14 by the gap formed between contacts 13, 14.

The particle detection sensor 6 comprises a magnet 15 which generates a magnetic field F within the gearbox section 3. As an example, the magnet 15 may be configured as a permanent magnet or as an electromagnet. The magnet 15 is preferably arranged between the two contacts 13, 14 so that a ferromagnetic particle arranged in the gearbox section 3 is moved into a measuring area M of the particle detection sensor 6 due to the magnetic interaction. In this regard, the measuring area 5 should be understood to be the area of the particle detection sensor 6 at which the ferromagnetic particles can make contact with the contacts 13, 14 or accumulate thereon.

FIGS. 3 to 5 each show the detection of different particle sizes or numbers by the particle detection sensor 6, as comprises already been described in FIG. 2. In a detection state, at least one particle 17 is arranged between the two contacts 13, 14, wherein the presence of one or more particles 17 can be detected based on an electrical parameter, in particular a change in impedance, between the two contacts 13, 14. Preferably, depending on the particle size and/or number, an electrically conductive bridge 16 is formed by one or more particles 17, which bridges the two contacts 13, 14 so that the circuit is closed.

The measured value output by the particle detection sensor 6 is evaluated with the aid of an evaluation criterion stored in the evaluation module 10 in order to determine a measure of the criticality of the flight operation. In this regard, the electrical parameter may, for example, be an ohmic resistance or a voltage, which are evaluated using Ohm's law. The measure of the criticality is determined on the basis of the detected particle size and/or number. As an example, a particle 17 with a small cross section comprises a higher electrical resistance than a particle 17 having a larger cross section, so that the particle size can be inferred from the electrical resistance. In addition, it is possible to draw conclusions as a function of time as to how many particles 17 have accumulated on the particle detection sensor 6 within a defined period of time.

As an example, the evaluation device 9 is configured to divide the measure of the criticality into three levels, wherein in a first level no criticality, in a second level a low criticality and in a third level a high criticality are indicated by the indicator device 12. In this regard, the levels are classified as a function of the determined particle size or number. As an example, the first level is indicated when no particles 17 are detected by the particle detection sensor 6. As an example, the second level is indicated when a single particle 17 with a maximum permissible particle size, for example less than or equal to 50 micrometres, is detected, as shown in FIG. 3. As an example, the third level is indicated when a particle 17 which is larger than the maximum permissible particle size is detected, as shown in FIG. 4, and/or when multiple particles 17 are detected at the particle detection sensor 6 within the specified time period, as shown in FIG. 5. As an example, the indicator device 12 may be configured to display the individual levels as different colours, for example as traffic light colours. As an example, the first level may be indicated as a green light signal, the second level as a yellow light signal and the third level as a red light signal. This will give the pilot the opportunity to decide whether an interruption to flight operations is necessary.

FIG. 6 shows, with the aid of a flow chart, decision-making logic for rating the criticality during a flight operation.

In a first step of the method, V1, the measured value from the particle detection sensor 6 is provided to the evaluation device 9 and optionally converted into a value that can be evaluated.

In a second step of the method, V2, the measured value is evaluated and classified into one of the three criticality levels S1, S2, S3.

In order to avoid a misinterpretation of the measured value, the measured value from the further particle detection sensor 7 and/or from the gearbox monitoring sensor 8 is added in a third step V3 of the method and classified into one of the three criticality levels S1, S2, S3 when the measured value from the particle detection sensor 6 is classified into the second or third criticality level S2, S3 in the second step of the method, V2.

In a fourth step of the method, V4, a warning signal is generated on the basis of the respective criticality level S2, S3 and provided to the indicator device 12, wherein the indicator device 12 is configured to indicate the respective criticality level S1, S2, S3 on the basis of the warning signal. In this regard, the indicator device 12 indicates the first level S1 until a particle 17 is detected by the particle detection sensor 6.

If, in the context of the second step of the method, V2, the measured value from the particle detection sensor 6 is classified into the second or third criticality level S2, S3 and in the context of the third step of the method, V3, the measured value from the further particle detection sensor 7 and/or from the gearbox monitoring sensor 8 is classified into the third criticality level S3, in the context of the fourth step of the method, V4, a warning signal to indicate the third criticality level S3 is generated.

If, on the other hand, in the context of the second step of the method, V2, the measured value from the particle detection sensor 6 is classified into the second or third level S2, S3 and in the context of the third step of the method, V3, the measured value from the further particle detection sensor 7 and/or from the gearbox monitoring sensor 8 is classified into the second criticality level S3, in the context of the fourth step of the method, V4, a warning signal to indicate the second criticality level S2 is generated. Thus, the pilot receives a qualitative statement about the state of the gearbox 2, whereupon the pilot can decide whether to terminate the flight or interrupt it on the basis of the indication.

### LIST OF REFERENCE NUMERALS

1 aircraft
2 gearbox
3 first gearbox section.
4 second gearbox section.
5 monitoring system
6 particle detection sensor
7 further particle detection sensor
8 gearbox monitoring sensor
9 evaluation device
10 evaluation module
11 rating module
12 indicator device
13 first contact
14 second contact.
15 magnet
16 conductive bridge
17 ferromagnetic particle
A spacing
F magnetic field
M measuring area
V1-V4 steps of the method
S1-S3 criticality levels

1. A method of monitoring a gearbox in an aircraft, wherein the gearbox comprises at least one gearbox section with at least one measuring area, wherein a first and a second electrical contact are arranged in the measuring area at a spacing from one another, wherein:
a magnetic field is generated in the gearbox section, wherein the magnetic field is adapted to move at least one ferromagnetic particle arranged in the gearbox section towards the measuring area, so that a bridge produced from one or more of the ferromagnetic particles is formed between the two electrical contacts;
a measured value is generated based on an electrical parameter between the two electrical contacts; and
a measure of the criticality of a flight operation is determined on the basis of the measured value.

2. The method of claim 1, wherein a warning signal is generated based on the measure of the criticality, and wherein a measure of the criticality of the flight operation is indicated based on the warning signal.

3. The method of claim 2, wherein the measure of the criticality is classified into at least two criticality levels, and wherein a respective warning signal is generated for each one of the criticality levels.

4. The method of claim 3, wherein no criticality is indicated in a first criticality level, a low criticality is indicated in a second criticality level and a high criticality is indicated in a third criticality level.

5. The method of claim 1, wherein the measure of the criticality is rated based on at least one further measured value which is detected independently of the measured value.

6. The method of claim 5, wherein a low criticality is indicated if the measured value and/or the further measured value is classified into the second criticality level, and in that a high criticality is indicated when both the measured value and/or the further measured value are classified into the third criticality level.

7. The method of claim 5, wherein the further measured value is output upon detection of at least one further ferromagnetic particle in a further measuring area of the gearbox and/or is generated upon a change of a gearbox parameter of the gearbox.

8. The method of claim 1, wherein the electrical parameter is an ohmic resistance and/or a voltage.

9. A monitoring system for a gearbox in an aircraft, with at least one particle detection sensor for detecting ferromagnetic particles in a measuring area,
wherein the particle detection sensor is arranged in a gearbox section of the gearbox,
wherein the particle detection sensor comprises two electrical contacts which are spaced apart from each other,
wherein the particle detection sensor comprises a magnet for generating a magnetic field, wherein the magnet is configured to move a ferromagnetic particle arranged in the gearbox section into a measuring area of the particle detection sensor, so that an electrically conductive bridge is formed between the two electrical contacts from one or more of the ferromagnetic particles,
wherein the particle detection sensor is configured to output a measured value on the basis of an electrical parameter between the two electrical contacts,
with an evaluation device for evaluating the measured value, and
wherein the evaluation device is configured to determine a measure of the criticality of the flight operation by evaluating the measured value.

10. The monitoring system of claim 9, further including at least one further particle detection sensor for detecting ferromagnetic particles in a further measuring area of the gearbox, wherein the further particle detection sensor is arranged in the gearbox section or in a further gearbox section, wherein the further particle detection sensor is configured to output a further measured value upon detection of at least one further ferromagnetic particle, wherein the evaluation device is configured to rate a measure of the criticality of the flight operation based on the measured value and the further measured value.

11. The monitoring system of claim 9, further including at least one gearbox monitoring sensor for detecting a gearbox parameter of the gearbox, wherein the gearbox monitoring sensor is configured to output the gearbox parameter as a further measured value, and wherein the evaluation device is configured to rate a measure of the criticality of the flight operation based on the measured value and the gearbox parameter.

12. The monitoring system of claim 9, further including an indicator device, wherein the indicator device is configured to indicate the criticality of the flight operation.

13. The monitoring system of claim 9, wherein a V-shaped gap is formed between the two contacts, the gap being adapted to accumulate a plurality of the ferromagnetic particles.

14. An aircraft, further including a monitoring system of claim 9.
